(19) United States
(12) Patent Application Publication
(10) Pub. No.: US 2002/0076806 A1
(43) Pub. Date: Jun. 20, 2002

(54) Sample injector system and method

(75) Inventor: Ezra Van Gelder, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: DNA Sciences, Inc., Fremont, CA (US)

(21) Appl. No.: 09/960,867

(22) Filed: Sep. 21, 2001

Related U.S. Application Data
(60) Provisional application No. 60/234,449, filed on Sep. 21, 2000.

(51) Int. Cl. C12M 1/34; G01N 33/53

(52) U.S. Cl. 435/287.2

(57) ABSTRACT

The present invention provides microfluidic systems and associated methods which allow material samples to be injected into an analysis channel independently of analysis techniques to reduce time required for testing. Such systems include an injector comprising channels which allow sample material to be loaded and injected into the analysis channel without interruption of analysis of the samples. Loading of the sample is performed within the microfluidic system without crossing or entering the analysis channel. The sample is then injected into the analysis channel at a desired time for testing or analysis. Thus, preparation time is significantly reduced so that overall testing time is largely dependent on actual analysis time. This is of particular import when a large number of samples are to be analyzed. In addition, the present invention provides for selection of a desired portion of the sample material for injection into the analysis channel, reducing possible bias in sample selection and providing greater control over the characteristics of the sample used.

### CROSS-REFERENCES TO RELATED APPLICATIONS

The present application claims the benefit of priority from U.S. Provisional Patent Application Serial No. 60/234,449 filed Sep. 21, 2000 (Attorney Docket No. 019553-003500), the full disclosure of which is incorporated herein by reference.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

1. Field of the Invention

The present invention relates generally to methods, systems and devices for use in the injection of microquantities of sample material into a conduit having small dimensions. In particular, the present invention provides microfluidic devices having a system of channels for injecting sample material into a channel for analysis. Typically, such sample material is biological and is moved through the channels by electric forces.

There is a need for reliable systems and devices capable of providing for the rapid injection of the components contained in microquantities of biological samples in order for the most recent advances in, separation and detection technology to be commercially viable and fully available for use in research and the diagnosis of disease. There is a particular need for devices and methods for analyzing genetic materials such as DNA, because variations in DNA can be associated with various genetic disorders.

Much of the success of modern molecular biology can be attributed to the development of reliable methods for the chemical structural analysis of nucleic acids. Determining the nucleotide sequence of DNA (deoxyribonucleic acids) and RNA (ribonucleic acid) is essential to recombinant DNA technology which aims to alter the genes of microorganisms so as to ultimately produce human proteins (drugs) such as interferon, growth hormone, insulin, etc. DNA sequencing information is also useful in developing plant strains that are resistant to adverse environmental conditions or disease. DNA analysis is also an effective approach for the detection and identification of pathogenic microbes and is essential to the identification of genetic disorders. The ability to detect DNA with clinical specificity entails high-resolution separation of RNA or DNA fragments, appropriate labeling chemistry for such fragments, and the adaption of high sensitivity sensors that are specific for the labeling chemistry employed.

The acquisition of such chemical and biochemical information requires expensive equipment, specialized laboratories and highly trained personnel. For this reason, laboratory testing is done in only a fraction of circumstances where acquisition of chemical information would be useful. A large proportion of testing in both research and clinical situations is done with crude manual methods that are characterized by high labor costs, high reagent consumption, long turnaround times, relative imprecision and poor reproducibility.

Many workers have attempted to solve these problems by creating integrated laboratories systems. Conventional robotic devices have been adapted to perform pipetting, specimen handling, solution mixing, as well as some fractionation and detection operations. More successful have been automated clinical diagnostic systems for rapidly and inexpensively performing a small number of applications such as clinical chemistry tests for blood levels of glucose, electrolytes and gases.

Recently, miniature components have been developed, particularly molecular separation methods and microvalves. One prominent field susceptible to miniaturization is capillary electrophoresis. Capillary electrophoresis has become a popular technique for separating charged molecular species in solution. It is known that fluids may be propelled through conduits by electro-osmotic force. Electro osmotic pressure is the consequence of charge buildup on the conduit surface. The buffer solution supplies the mobile counter ion to neutralize the surface charge and is the potential energy equivalent of the electro osmotic pressure. The application of an external voltage will cause a discharge via the mobile ions, resulting in an electro-kinetic current. The discharge of ions causes the fluids in the conduit to flow. For example, the fluid flow is in the direction of the negative pole of the electric field when the counter ions are cations. The fluid flow direction is controlled by the magnitude of the applied voltage, its polarity, the surface charge, the channel dimensions and the viscosity of the medium.

The technique of capillary electrophoresis is performed in small capillary tubes to reduce band broadening effects due to thermal convection and hence improve resulting power. The capillary tubes typically comprise fused silica capillaries with nominal dimensions of 1 meter length and 80-100 μm diameter. The voltage used to electro-osmotically drive the fluids through such capillaries at a rate of approximately 0.2 microliters per minute is approximately 200 volts/cm. The small size of the capillaries implies that minute volumes of materials, on the order of nanoliters, must be handled. Typically, these volumes samples of material are injected into a separation capillary tube or channel for separation by electrophoresis.

Electrophoresis is an analytical technique to separate and identify charged particles, ions, or molecules. It involves the imposition of electric fields to move charged species in a liquid medium. Molecules are separated by their different mobilities under an applied electric field. The mobilities variation derives from the different charge and frictional resistance characteristics of the molecules. When a mixture containing several molecular species is introduced into the electrophoretic separation channel and an electric field is applied, the different charge components migrate at various speeds in the system leading to the resolution of the mixture. Bands appear, depending on the mobilities of the components.

Capillary electrophoresis has further been miniaturized by technology originally developed in the semiconductor electronics industry to develop microfluidic systems for the separation of biological samples. The term “microfluidic” as typically used refers to a device created using techniques such as photolithography and wet chemical etching to fabricate channels and/or wells in a substrate or wafer which may be as small as a micron or submicron in scale. Early work in this field, particularly the fabrication of microfluidic devices in silicon and glass substrates, is described in Manz et al., Trends in Anal. Chem., 10:144-149, 1990, and Manz et al., Adv. in Chromatog., 33:1-66, 1993. These references are incorporated herein by reference in their entirety for all purposes.

In most existing microfluidic devices designed for sample analysis, samples are moved through the micro-channel network by application of a force to the micro-channels. Most commonly, samples are transported through the micro-channels by applying and varying multiple electric fields. The aim is to transport the sample to an analysis channel where the sample is analyzed by electrophoresis or other methods. In many situations, it is desirable to analyze as many discrete samples as possible in the shortest amount of time. This is limited by the time in which it takes to analyze a sample, the number of samples which can be analyzed simultaneously and the time in which it takes to load or inject the samples in the analysis channel, to name a few.

Thus there exists a need for reliable, low-cost, automated analytical methods and devices that allow rapid injection, separation and detection of microquantities of sample material for use in the research and diagnosis of disease. Specifically, methods and devices for injecting material samples into an analysis channel quickly, consistently, and without contamination. At least some of these objectives are met by the inventions described hereinbelow.

2. Description of the Background Art

An analytical separation device is discussed by Pace, U.S. Pat. No. 4,908,112, in which a capillary sized conduit is formed by a channel in a silicon semiconductor wafer and the channel is closed by glass plates. Electrodes are positioned in the channel to activate the motion of liquid through the conduit by electroosmosis.

Microchip laboratory systems and methods are discussed by Ramsey, U.S. Pat. Nos. 6,033,546; 6,010,608; 6,010,607; 6,001,229; 5,858,195; and 5,858,187, providing fluid manipulations for a variety of applications, including sample injection for microchip chemical separations.

Microfluidics devices which incorporate improved channel and reservoir geometries are discussed by Dubrow et al., U.S. Pat. Nos. 6,153,073 and 6,235,175. Likewise, a multi-port device which includes a substrate having a novel channel configuration is described by Chow et al., U.S. Pat. Nos. 5,965,410 and 6,174,675.

Methods and devices related to the movement of molecules with electro-osmotic flow systems is discussed by Nikiforov et al., U.S. Pat. No. 5,964,995, and Soane et al., U.S. Pat. No. 6,093,296. Further, a device and method for performing spectral measurements and flow cells with spatial resolution is described by Weigl et al., U.S. Pat. No. 6,091,502.

### BRIEF SUMMARY OF THE INVENTION

The present invention provides microfluidic systems and associated methods which allow material samples to be injected into an analysis channel independently of analysis techniques to reduce time required for testing. Such systems include an injector comprising channels which allow sample material to be loaded and injected into the analysis channel without interruption of analysis of the samples. Loading of the sample is performed within the microfluidic system without crossing or entering the analysis channel. The sample is then injected into the analysis channel at a desired time for testing or analysis. Thus, preparation time is significantly reduced so that overall testing time is largely dependent on actual analysis time. This is of particular import when a large number of samples are to be analyzed. In addition, the present invention provides for selection of a desired portion of the sample material for injection into the analysis channel, reducing possible bias in sample selection and providing greater control over the characteristics of the sample used.

In a first aspect of the present invention, a microfluidic system is provided comprising a structure having an analysis channel and various additional channels which provide for loading and injection of a sample into the analysis channel. These additional channels include an injection channel, a loading channel and a waste channel. The injection channel intersects the analysis channel at a three-way first intersection. Thus, the injection channel typically intersects the analysis channel in a “T” configuration so that a three-way intersection is formed between the channels. Usually, the injection channel does not cross the analysis channel as in a four-way intersection. The loading channel and waste channel intersect the injection channel at a second intersection. The loading channel and waste channel intersect so that sample moving from the loading channel may pass through the second intersection to the waste channel.

In a second aspect of the present invention, the system further comprises means for moving sample material through the channels. Typically, sample is moved by electric forces. Since the channels are filled with a fluid or gel, electric forces can be transmitted through the channels. Electric forces are generated by independent voltage sources or by a selectable voltage controller in contact with the fluid or gel. This is most easily achieved by contacting wells which are in fluid connection with the channels. In most embodiments, a sample well is fluidly connected to the loading channel and a waste well is fluidly connected to the waste channel. The sample well is used for loading sample into the microfluidic system. The waste well is used for collecting waste sample material for disposal or removal from the system. By positioning at least one electrode in each the sample well and the waste well, a voltage differential can be applied across the channels therebetween. Depending on the voltage differential applied, the sample material can be drawn from the sample well toward the waste well.

Most embodiments additionally include a first well and a second well, each fluidly connected to the analysis channel. Typically, each of these wells is located at opposite ends of the analysis channel. By applying a voltage differential between the first and second well, separation techniques may be performed in the analysis channel. Such a voltage differential may be applied with the use of electrodes positioned in the wells as mentioned above. In addition, a voltage differential may be applied between the first well and/or second well and the other wells to control movement of sample material through the channels of the microfluidic system.

In another aspect of the present invention, methods are provided for moving sample material through the channels, including injection of the material into the analysis channel. To begin, sample material is drawn from the sample well toward the waste well. This may be achieved by applying a voltage differential between the sample well and waste well. The sample migrates through the loading channel to the second intersection, the intersection of the loading channel, injection channel and waste channel. The fastest moving components of the sample, typically the smallest components, will reach the intersection first. If it is desired to analyze a portion of the sample material having components of more equally varied size or motility, the sample is allowed to migrate beyond the second intersection into the waste channel. Once a desired portion of sample material reaches the second intersection, movement toward the waste well is halted.

The sample material is then moved through the injection channel to the first intersection, the intersection of the injection channel with the analysis channel. This may be achieved by applying a voltage differential between the first well or second well and the sample well. In this step, the desired portion of sample material located at the second intersection is drawn to the first intersection as additional sample material follows behind. Generally, the additional sample material contains a similar or identical assortment of components since the sample material is often consistent after the initial portion of material passes through to the waste channel. The sample material may continue to move beyond the first intersection and into the analysis channel until a desired quantity of sample material enters the analysis channel.

Sample material that has not entered the analysis channel is then removed by drawing the excess material back through the injection channel to the waste well. This may be achieved by applying a voltage differential between the sample well and waste well. The portion of material that remains in the analysis channel is termed a “plug” and will then be analyzed by electrophoresis or other suitable methods.

It may be appreciated that sample material may be moved through the channels by other means, such as by pressure differentials. Pressure differentials may be generated by applying a vacuum to a well to create a lower pressure. This causes the sample to move through the channels toward the area of lower pressure. Alternatively, pumps or related devices could be used to create a higher pressure within a well or channel thereby forcing the sample away from the higher pressure. And in some cases, it may be possible to move the sample through the channels by gravity flow. Thus, although most examples will be described in terms of electric forces, other types of forces may be utilized and any of combination of these forces may be used.

The system and methods of the present invention provide advantages to current methods of injection of samples for analysis techniques. By loading and preparing the sample within the loading channel, waste channel and injection channel, the analysis channel may be utilized for uninterrupted analysis of sample material during these steps. Other injection systems require interruption of analysis methods during loading of the sample which costs valuable testing time. In addition, the system and methods of the present invention allow multiple samples to be loaded within the analysis channel for simultaneous and/or sequential analysis. This also reduces testing time. Further, such loading and preparation within the loading channel and waste channel allows for selection of a desired portion of sample material. As described, this portion of material is selected and moved to through the injection channel to the analysis channel for future analysis. Other injection systems load sample material directly from the sample well to the analysis channel. This does not allow the user control over the characteristics of the sample used.

Other objects and advantages of the present invention will become apparent from the detailed description to follow, together with the accompanying drawings.

### BRIEF DESCRIPTION OF THE DRAWINGS

FIG. 1 is a schematic illustration of a preferred embodiment of the microfluidic system of the present invention.

FIGS. 2A-2E are schematic illustrations of an injection sequence for loading sample material into the analysis channel.

FIG. 3 illustrates the capability of repeating the injection sequence while the plug of sample material is analyzed in the analysis channel.

FIG. 4 illustrates the loading of multiple samples into the analysis channel.

FIGS. 5A-5C illustrate a prior art system and method of injection utilizing a T-shaped configuration.

FIGS. 6A-6C illustrate a prior art system and method of injection utilizing a cross-shaped configuration

FIGS. 7A-7D illustrate additional embodiments of the microfluidic system of the present invention which involve loading and waste channels having a variety of configurations.

FIG. 8 illustrates an embodiment of the present invention having more than one injection channel intersecting the analysis channel.

FIG. 9 illustrates an embodiment of the present invention having more than one set of loading and waste channels intersecting the injection channel.

### DETAILED DESCRIPTION OF THE INVENTION

The present invention generally provides microfluidic devices or systems which incorporate improved sample injection systems, as well as methods of using these devices or systems in the loading, injection, testing, analysis or other manipulation of fluid suspended sample materials.

I. General Overview

As mentioned, the microfluidic system of the present invention incorporates an improved sample injection system. Sample injection systems are used to inject one or more discrete portions or “plugs” of fluid samples into an analysis channel wherein the samples are tested or analyzed. Such analysis may comprise electrophoresis wherein the analysis channel may be termed an electrophoretic separation channel.

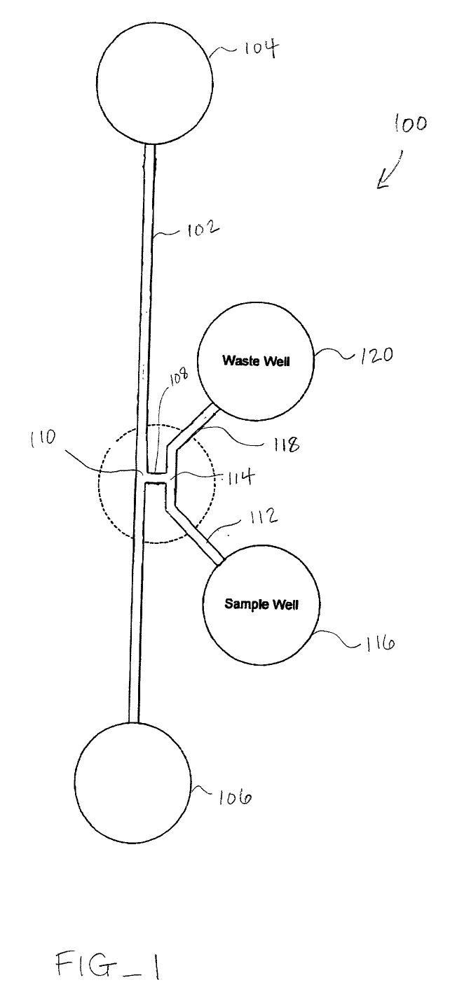

FIG. 1 schematically illustrates a preferred embodiment of the microfluidic system of the present invention having an injection system in the shape of an “H”. Thus, the system may be referred to as an H-injector. Here, the microfluidic system 100 comprises an analysis channel 102 which spans between a first well 104 and a second well 106 as shown. In some embodiments, the analysis channel 102 has a length of approximately 7 cm. The system 100 further comprises an injection channel 108 which intersects the analysis channel 102 at a three-way first intersection 110. The injection channel 108 is relatively short, such as 1-2 mm in length. The injection channel 108 may intersect the analysis channel 102 at any suitable angle, including a 90 degree angle as shown. The system 100 further comprises a loading channel 112 which intersects the injection channel 108 at a second intersection 114. The loading channel 112 receives sample material from a sample well 116 which is fluidly connected with the loading channel 112 as shown. Further, the system comprises a waste channel 118 which also intersects the injection channel 108, either at the second intersection 114 as shown or at another point of intersection along the injection channel 108. The waste channel 118 is fluidly connected with a waste well 120 for receiving waste sample fluid from the waste channel 118. In some embodiments, the sample well 116 and waste well 120 are approximately 1 cm apart, however such distance is dependent on the arrangement of the channels. The loading channel 112 and waste channel 118 may intersect the injection channel 108 at any suitable angle to the injection channel, including a 90 degree angle as shown. Thus, the angles with which the channels intersect are not a critical feature of the invention.

Movement of the sample through the channels is achieved by any suitable means, such as by electric forces or pressure differentials. Electric forces may be generated by a selectable voltage controller which applies a desired voltage level, including ground, to each well 104, 106, 116, 120. The voltage controller may utilize multiple voltage dividers and relays to obtain the selectable voltage levels. The voltage controller is electrically connected to each of the wells 104, 106, 116, 120 by an electrode which is positioned or fabricated within each of the wells. A description of how this is accomplished is set forth in PCT publication WO 96/04547 to Ramsey, and is incorporated herein by reference in its entirety for all purposes. It may be appreciated that multiple independent voltage sources may be used in a similar manner.

When voltages are applied to wells at opposite ends of the channel, a voltage differential is created across the channel. Charged material within the channel is drawn toward a well to which it is more strongly attracted. For example, when the sample material itself is charged, such as DNA fragments (negatively charged in the case of electrophoresis), the sample material will move through the fluid or gel filled channels toward, in this case, a positively charged well when a field is applied. Under other circumstances, electric fields can induce electro-osmotic flow which can carry positive, neutral or negative ions at different speeds through a channel. However, overall, when a voltage differential is applied between two wells, the material is more strongly attracted to one of the wells. To this end, throughout this application, areas to which a material is more attracted will be referred to as positive or positively charged and areas to which a material is less attracted will be referred to as negative or negatively charged. This does not imply positive or negative polarity. By manipulating the voltages, sample material may be transported through the channels in a controlled manner.

Alternatively, pressure differentials may be used to move sample material through channels with the use of vacuums, pumps or various other devices. These devices may be connected to each of the wells 104, 106, 110, 120 by mechanical attachments. When a pump is applied to the sample well 116, for example, sample material will move through the channels away from the sample well. Sample material moving through the loading channel 112 toward the second intersection 114 may continue moving through the injection channel 108 and/or waste channel 118 depending on the pressures within these channels. Pumps may be applied to other wells, such as the first well 104 and second well 106 to force the material toward the waste well 120. Alternatively or in addition, a vacuum may be applied to the waste well 120 to draw material toward the waste well 120. In this case, the vacuum may additionally serve to remove material from the waste well 120. It may be appreciated that both pressure differentials and voltage differentials may be used to move material through the system, either simultaneously or sequentially. Thus, a variety of devices may be used singly or in combination to achieve similar results.

II. Structure

The microfluidic systems comprise a structure, within which channels and/or wells are disposed, and a coverplate which is overlaid and bonded to the structure thereby defining and sealing the channels and/or wells of the structure.

The structure is typically planar, i.e. substantially flat or having at least one flat surface, and may be fabricated from any suitable solid or semi-solid substrate or combination of materials. Often, the planar substrates are manufactured using solid substrates common in the fields of microfabrication, such as silica-based substrates, glass, quartz, silicon or polysilicon, as well as other substrates, such as gallium arsenide. Alternatively, polymeric substrate materials may be used to fabricate the devices of the present invention, including polydimethylsiloxanes (PDMS), polymethylmethacrylate (PMMA), polyurethane, polyvinylchloride (PVC), polystyrene polysulfone, polycarbonate, polymethylpentene, polypropylene, polyethylene, polyvinylidine fluoride, ABS (acrylonitrilebutadiene-styrene copolymer), and the like. These materials may be rigid, semi-rigid, or nonrigid, opaque, semi-opaque, or transparent depending upon the use for which the material is intended. For example, devices which include an optical or visual detector are generally fabricated, at least in part, from transparent materials to facilitate detection of sample material by the detector. Other components of the device, especially the cover plate, can be fabricated from the same or different materials depending on the particular use of the device, economic concerns, solvent compatibility, optical clarity, mechanical strength and other structural concerns.

The channels are typically fabricated into one surface of the planar substrate as grooves, furrows or troughs. In addition, the channels often intersect with wells or reservoirs which are used for loading or removing sample material. Such wells are typically formed as depressions in the surface and are fabricated in a manner similar to that of the channels. This may be achieved by common microfabrication techniques, such as photolithographic techniques, wet chemical etching, micromachining, i.e. drilling, milling and the like. In the case of polymeric materials, injection molding or embossing methods may be used to form the substrates having the channels described herein. In such cases, original molds may be fabricated using any of the above materials and methods.

The size and shape of the channels and reserviors or wells is generally not critical. The channels have essentially any shape, including, but not limited to, semi-circular, cylindrical, rectangular and trapezoidal. The depths of the channels can vary, but tends to be approximately 10 to 100 microns, most typically about 35-50 microns. As a result of the manufacturing process used, the channels are commonly approximately twice as wide as they are deep. Thus, the channels tend to be 20 to 200 microns wide. However, the actual width is not critical.

After forming the channels and wells, the cover plate may be attached to the substrate by a variety of means, including, for example, thermal bonding, adhesives or a natural adhesion between the substrate and cover plate, he and as may be possible with the use of certain substrates such as glass, or semi-rigid and non-rigid polymeric substrates. The cover plate may additionally be provided with access ports for introducing the various liquids into the channels or reservoirs. It may be appreciated that the coverplate serves to form closure to the channels and wells so that they are not open structures. Thus, throughout this application the terms channel, well, reservoir and others related to such structures are synonymous with closed channel, well, reservoir, etc.

III. Samples

The microfluidic devices and methods provided by the current invention can be used in a wide variety of separation-based analyses, including sequencing, purification, and analyte identification applications for clinical, environmental, quality control and research purposes. Consequently, the type of samples that can be analyzed is equally diverse. Representative sample types include bodily fluids, environmental fluid samples, or other fluid samples in which the identification and/or isolation of a particular compound or compounds is desired.

The source of the sample may be blood, urine, plasma, cerebrospinal fluid, tears, nasal or ear discharge, tissue lysate, saliva, biopsies, and the like. Examples of the types of compounds actually analyzed include, for instance, small organic molecules, metabolites of drugs or xenobiotics, peptides, proteins, glycoproteins, oligosaccharides, oligonucleotides, DNA, RNA, lipids, steroids, cholesterols, and the like. The amount of sample initially injected into a sample reservoir within the structure can be varied, and can be less than 1 microliter in volume.

The system and methods of the invention are particularly useful for detecting primer extension products resulting from analysis of single nucleotide polymorphisms (SNPs) in target samples. A SNP usually arises due to substitution of one nucleotide for another at a polymorphic site. A purine may be replaced by another purine, termed a transition, or a purine may be replaced by a pyrimidine or vice versa, termed a transversion. SNPs can also arise from a deletion of a nucleotide or an insertion of a nucleotide relative to a reference allele. Thus, SNPs are a particular type of polymorphism wherein polymorphism refers to the occurrence of two or more genetically determined alternative sequences or alleles in a population. The polymorphic marker or site is the locus at which divergence occurs. Preferred markers have at least two alleles, each occurring at frequency of greater than 1%, and more preferably greater than 10% or 20% of a selected population. As stated, a polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms.

To analyze SNPs, single base extension methods are used as described by e.g., U.S. Pat. Nos. 5,846,710, 6,004,744, 5,888,819 and 5,856,092. In brief, a primer that is complementary to a target sequence is hybridized such that the 3′ end of the primer is immediately adjacent to but does not span a site of potential variation in the target sequence. That is, the primer comprises a subsequence from the complement of a target polynucleotide terminating at the base that is immediately adjacent and 5′ to the polymorphic site. The hybridization is performed in the presence of one or more labeled nucleotides complementary to base(s)that may occupy the site of potential variation. For example, for a biallelic polymorphisms two differentially labeled nucleotides can be used. For a tetraallelic polymorphisms four differentially labeled nucleotides can be used. In some methods, particularly methods employing multiple differentially labeled nucleotides, the nucleotides are dideoxynucleotides. Hybridization is performed under conditions permitting primer extension if a nucleotide complementary to a base occupying the site of variation in the target sequence is present. Extension incorporates a labeled nucleotide thereby generating a labeled extended primer. If multiple differentially labeled nucleotides are used and the target is heterozygous then multiple differentially labeled extended primers can be obtained. Extended primers are detected providing an indication of which bas(es) occupy the site of variation in the target polynucleotide. The systems and methods of the present invention may be used to inject and then analyze the extended primers.

Alternatively, SNPs can be detected by allele-specific primer extension. An allele-specific primer hybridizes to a site on target DNA overlapping a polymorphism and only primes amplification of an allelic form to which the primer exhibits perfect complementarily. See Gibbs, Nucleic Acid Res. 17, 2427-2448 (1989). This primer is used in conjunction with a second primer that hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. In some methods, the mismatch is included in the 3′-most position of the oligonucleotide aligned with the polymorphism because this position is most destabilizing to elongation from the primer. See, e.g., WO 93/22456. Primer extension products may be analyzed using the apparatus and methods of the present invention.

IV. Injection Sequence for Loading Sample

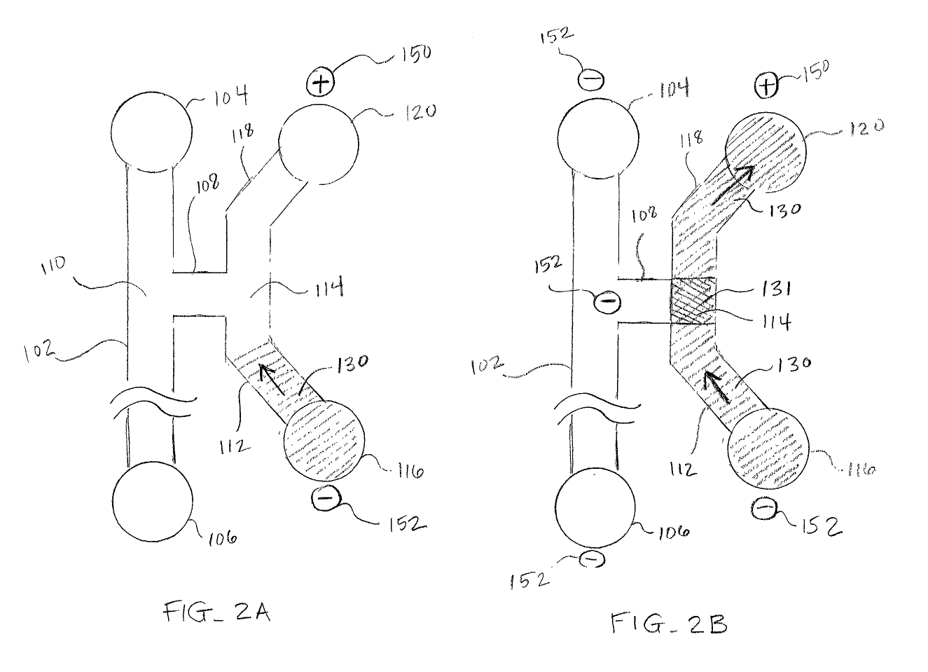

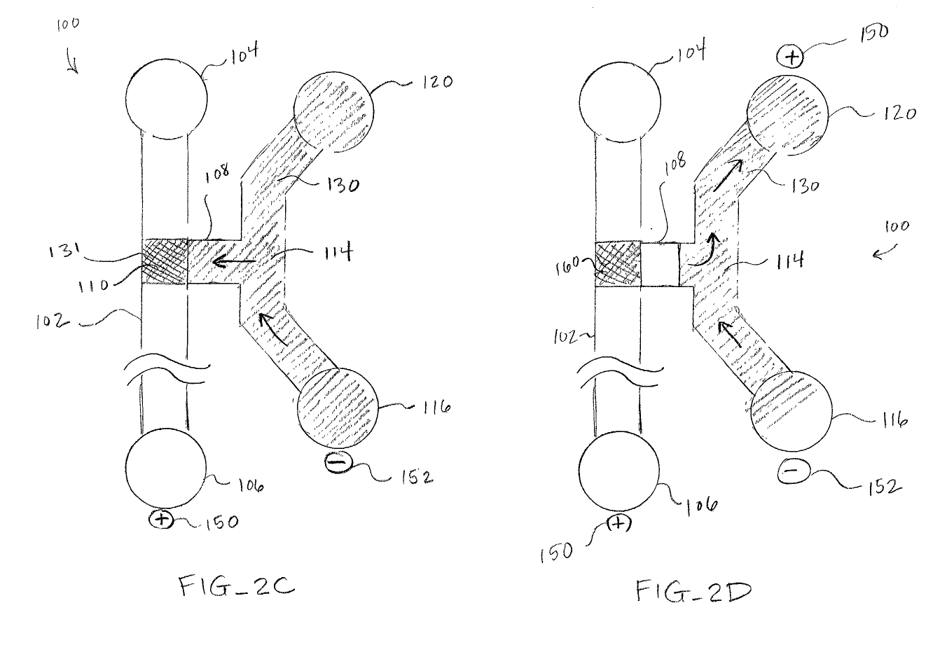

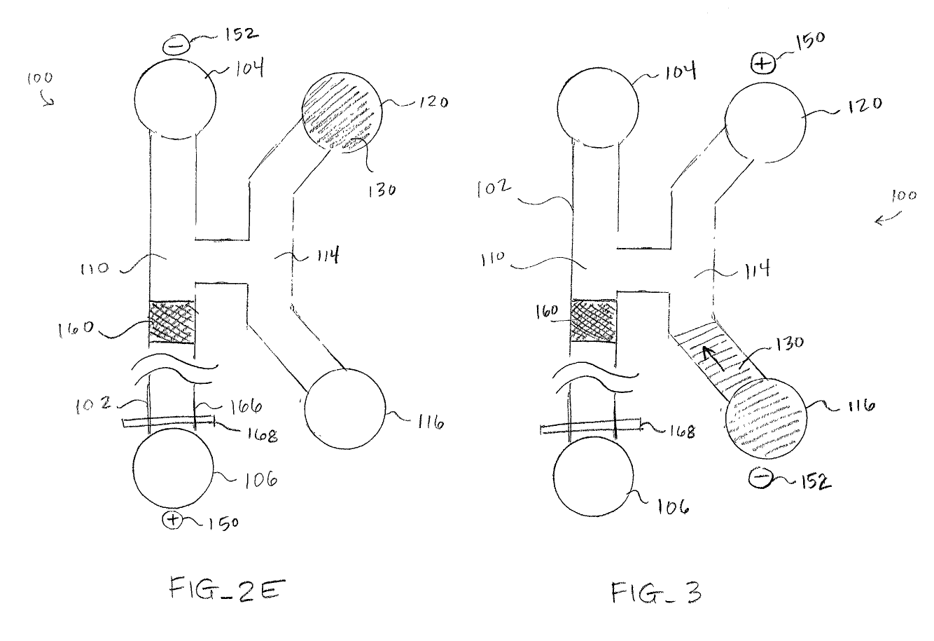

FIGS. 2A-2E schematically illustrate an injection sequence for loading sample material 130 (indicated by shading and directional arrows) into the analysis channel 102 with the use of the H-injector. Referring to FIG. 2A, sample material 130 is loaded in the sample well 116 by standard methods. A loading force is applied between the sample well 116 and waste well 120 to draw the sample material 130 from the sample well 116 toward the waste well 120, as indicated by the directional arrow. Such a loading force may comprise a voltage differential. For example, the sample material 130 is attracted to the waste well 120 (signified by positive symbol 150) and away from the sample well 116 (signified by negative symbol 152) Such a voltage differential may be in the range of 200-400 volts.

Since the sample material 130 is comprised of components which migrate at various speeds, the portion of sample material 130 which is first to reach the second intersection 114 will be highly concentrated with fast migrating components. In instances where it is desired to analyze portions of sample material 130 having a more diverse spectra of components, the material 130 can migrate past the second intersection to or toward the waste well 120, as illustrated in FIG. 2B. This can continue until a portion of desired sample material 131 (material having a desired concentration of specific components) reaches the second intersection 114, as depicted by double hatch shading. The amount of time required for this migration depends on the material 130, the voltages applied and the time during which the material 130 is allowed to be transported. In other words, the voltages may be chosen and applied such that the material 130 is transported to or toward the waste well 120 at a desired speed until the desired sample material 131 arrives at the second intersection 114.

Typical migration times are 20-60 seconds, more typically 30 seconds. To assist in drawing the sample material 130 toward the waste well 120 and away from the injection channel 108, a voltage gradient may be applied between the first well 104 and second well 106 to create a repulsion at the first intersection 110 and within the injection channel 108.

Referring to FIG. 2C, an injection force is then applied to draw the desired sample material 131 at the second intersection 114 through the injection channel 108 and into the analysis channel 102 at the first intersection 110. This may be accomplished by applying a voltage differential between the sample well 116 and the second well 106. The voltage differential applied would typically be sufficient to create a voltage at the first intersection 110 which is 10-50 volts lower than the voltage at the second intersection 114. Such migration is typically accomplished in the range of approximately 1-10 seconds, typically 5-10 seconds. As shown, additional sample material 130 follows as indicated by directional arrows. Thus, if the sample material 130, 131 is allowed to migrate further along the analysis channel 102, the quantity of material 130, 131 within the analysis channel 102 will increase. The speed and control of migration may be manipulated by the application of voltage differentials across other points in the system, such as the first well 104 and the waste well 120.

Referring now to FIG. 2D, a withdrawal force is then applied to draw any excess sample material 130 back through the injection channel 108 and waste channel 118 to the waste well 120, as indicated by directional arrows. In addition, any material 130 within the loading channel 112 and sample well 116 may also be transported to the waste well 120. This may be achieved by applying a voltage differential between the sample well 116 and the waste well 120. Material remaining within the analysis channel 102 is termed a “plug” 160 which will later be analyzed. The more material that was allowed to enter the analysis channel 102, the longer the length of the plug 160. Additional voltage differentials may be applied throughout the system to maintain the plug 160 within the analysis channel 102 while the remaining material 130 is transported to the waste well 120.

Referring to FIG. 2E, the plug 160 resides in the analysis channel 102 ready for analysis while the remainder of material 130 is transported to the waste well 120. During or after such transport, the plug 160 may be analyzed by applying an analysis force. In this example, the analysis channel 102 comprises an electrophoretic separation channel 166 wherein the plug 160 is analyzed by electrophoretic separation. To enhance separation of the components in the plug 160, a separation material is preferably included within the separation channel 166. A variety of different separation materials can be utilized. In general, any chromatographic material could be utilized, including, for example, absorptive phase materials, ion exchange materials, affinity chromatography materials, materials separating on the basis of size, as well as those separating on the basis of some functional group. A variety of electrophoretic materials can also be used. Of particular utility are cellulose derivatives, polyacrylamides, polyvinyl alcohols, polyethylene oxides, and the like. Preferred electrophoretic media include linear acrylamide and hydroxyethyl cellulose, polyvinyl alcohol and polyethylene oxide. By judicious selection of the appropriate separation material, a separation can be achieved on the basis of a number of different parameters defining the plug components, such as charge, size, chemical characteristics, or combinations thereof.

To commence the separation, voltage differentials are applied between the first well 104 and the second well 106 to generate a controlled electric field between the wells 104,106. Such voltage differentials are approximately 1400 volts. The resulting electric field causes the components of the plug 160 to migrate. Faster migrating components separate from slower components forming bands. As the components migrate down the analysis channel 102, the components pass by a detector 168 which monitors the presence of various components within the plug 160. Various detectors may be used depending on the nature of the components being separated. For example, the detector 168 may be any other variety of optical or electrochemical detectors. For optical detectors, it is advantageous for the cover plate to be manufactured from a material which is optically transparent in the spectral range measured by the detector.

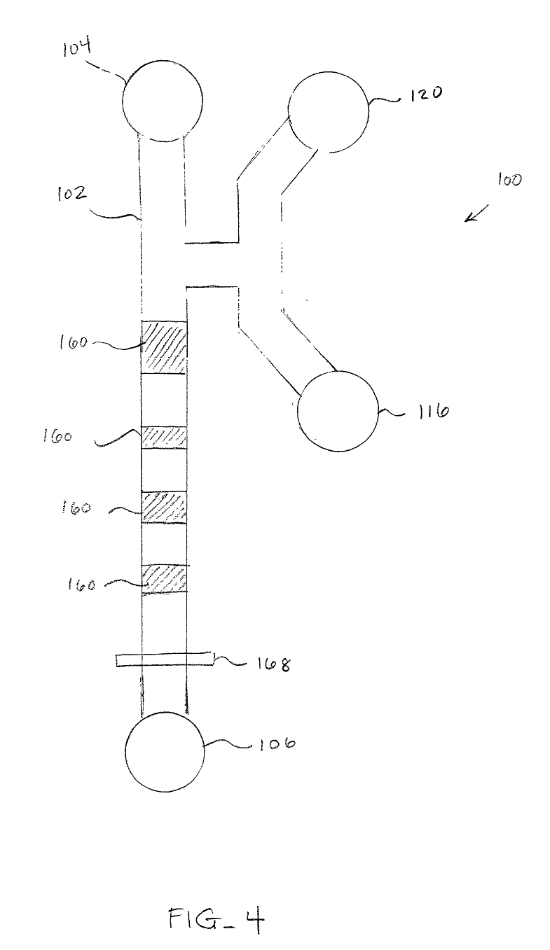

Referring to FIG. 3, during the analysis of the plug 160, the injection sequence may be repeated to load a second discrete plug of sample material into the analysis channel 102. New sample material 170 is loaded in the sample well 116 by standard methods. This may include removing portions of the previous sample from the sample well 116. As in FIG. 2A, voltage differentials are applied to the sample well 116 and the waste well 120 to transport the sample material 170 from the sample well 116 toward the waste well 120. The injection sequence may continue as previously shown in FIGS. 2B-2E. As shown in FIG. 4, this may result in a number of discrete plugs 160 being loaded in the analysis channel 102. The plugs 160 may be of a variety of sizes and material compositions. The plugs 160 may be sequentially or simultaneously analyzed. In addition, such analysis may ensue independently of the injection sequences.

It may be appreciated that the above described injection sequence illustrates an embodiment of the present invention and is not intended to limit the scope of the invention. For example, in FIG. 2E the sample material 130 may alternatively migrate through the analysis channel 102 toward the first well 104 if the voltage differentials were reversed. Likewise, the sample material may be neutrally charged and transported through the channels by movement of a charged buffer solution. The determination of whether the sample material 130 is to migrates toward the first well 104 or second well 106 depends upon the analysis to be undertaken. Typically, when the analysis involves electrophoresis, the analysis channel 102 includes a relatively long separation channel 166 with a detector 168. Obviously, sample material should be directed to the well on the opposite end of the separation channel, beyond the detector. Other analysis techniques may be used, such as involving a mass spectrometer. In this case, the analysis channel 102 may simply guide the sample material into the mass spectrometer. Thus, any number of embodiments exist utilizing the basic principles of the present invention.

Comparison with Prior Art Systems

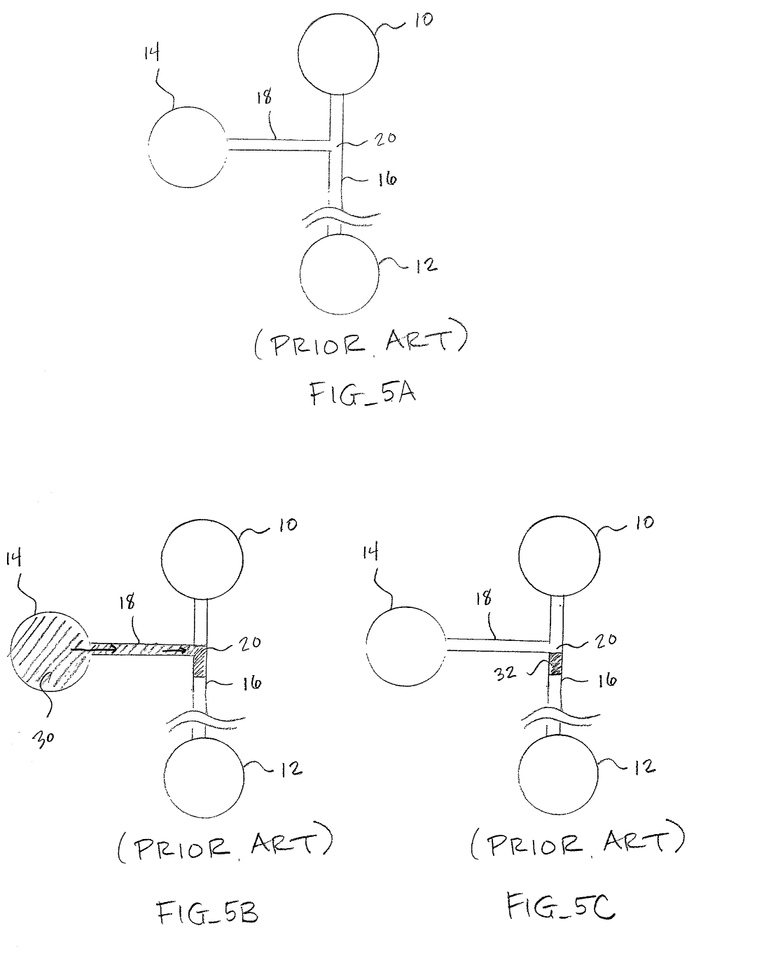

Prior art systems and methods of injecting sample material into a separation channel have a variety of shortcomings which are overcome by the present invention. FIGS. 5A-5C illustrate one such prior art system. Referring to FIG. 5A, a separation channel 16 fluidly connects a first reservoir 10 with a second reservoir 12. A connection channel 18 fluidly connects an input reservoir 14 with the separation channel at a T-intersection 20. FIGS. 5B-5C illustrate injection of sample into the separation channel for analysis. As shown in FIG. 5B, sample 30 loaded in the input reservoir 14 is drawn through the connection channel 18 (indicated by shading and directional arrows) and into the separation channel 16. This may be achieved by applying a voltage differential between the input reservoir 14 and the first or second reservoir 10, 12, in this example the second reservoir 12. It may be appreciated that other types of force may also move the sample through the channels. Once a sufficient quantity of sample material 30 has entered the separation channel 16, the excess material is removed leaving a plug 32 in the separation channel 16, as shown in FIG. 5C. One major drawback of this system and method is that the plug 32 will be comprised of components within the sample material 30 which are first to reach the separation channel 16. Typically such components are the shorter, more fast moving components. Consequently, the plug 32 is not a representative portion of the sample material 30.

The present invention overcomes such sample bias. As previously shown in FIG. 2B, the material 130 can migrate past the second intersection to or toward the waste well 120. This may continue until a portion of desired sample material 131 (material having a desired concentration of specific components) reaches the second intersection 114. As shown in FIG. 2C, the desired sample material 131 at the second intersection 114 is then drawn through the injection channel 108 and into the analysis channel 102 at the first intersection 110.

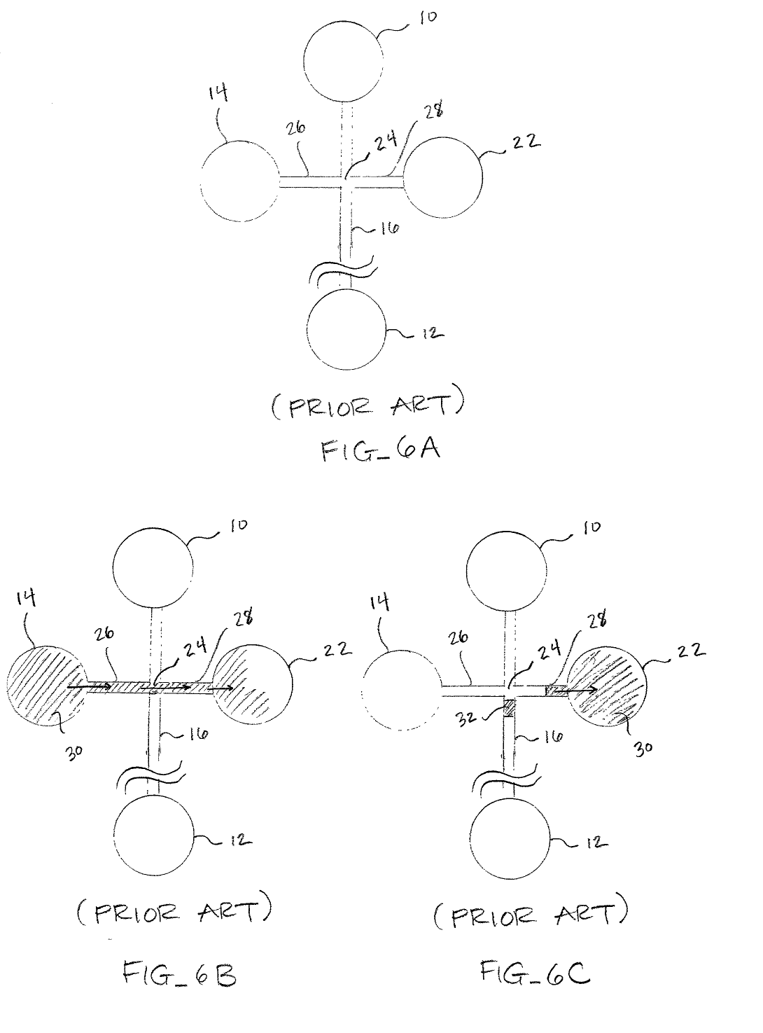

Other prior art systems which have been designed to overcome sample bias require steps of preparation, loading and injection of the sample which interfere with the analysis step. Thus, analysis must be interrupted during preparation, loading and injection of the sample which adds significant time to the testing period. One such system is illustrated in FIGS. 6A-6C. Referring to FIG. 6A, a separation channel 16 fluidly connects a first reservoir 10 with a second reservoir 12. A first connection channel 26 fluidly connects an input reservoir 14 with the separation channel 16. A second connection channel 28 fluidly connects an output reservoir 22 with the separation channel 16. The first and second connection channels 26, 28 may intersect the separation channel 16 at a cross-intersection 24 as shown, or the channels 26, 28 may intersect the separation channel 16 at two separate intersection points (not shown). In either case, the input reservoir 14 and output reservoir 22 reside on opposite sides of the separation channel 16. FIGS. 6B-6C illustrate injection of sample into the separation channel for analysis. As shown in FIG. 6B, sample 30 loaded in the input reservoir 14 is drawn through the first connection channel 26 (indicated by shading and directional arrows), through the separation channel 16 and into the output reservoir 16. This may be achieved by applying a voltage differential between the input reservoir 14 and the waste reservoir 22. Again, it may be appreciated that other types of force may also move the sample through the channels. The sample 30 continues moving until a desired portion of the sample resides within the cross-intersection 24. At this point, as shown in FIG. 6C, the material within the cross-intersection 24 is moved through the separation channel 16 forming a plug 32. This is generally achieved by applying a voltage differential between the first reservoir 10 and the second reservoir 12. The excess material is then moved to the output reservoir 22 for removal. Thus, sample analysis or separation within the separation channel 16 cannot be performed throughout the loading and injection steps since the undesired and excess material is crossing the separation channel 16 to reach the output reservoir 22. Consequently, the time required to perform these steps is additive with the time to perform the separation itself, compounding the total experiment time with each sample.

The present invention overcomes such time compounding. As previously shown in FIGS. 2A-2B, sample material 130 loaded in the sample well 116 is drawn toward the waste well 120, as indicated by the directional arrow, without crossing or interfering with the analysis channel 102. Thus, loading the sample and selecting a desired portion of the sample is performed simultaneously with performing analysis on samples present in the separation channel 102. Since the injection channel 108 is relatively short in length, the time required to inject the prepared sample into the separation channel 102 is minimal. This significantly reduces the total experiment time, particularly when loading numerous sample plugs.

Additional Embodiments

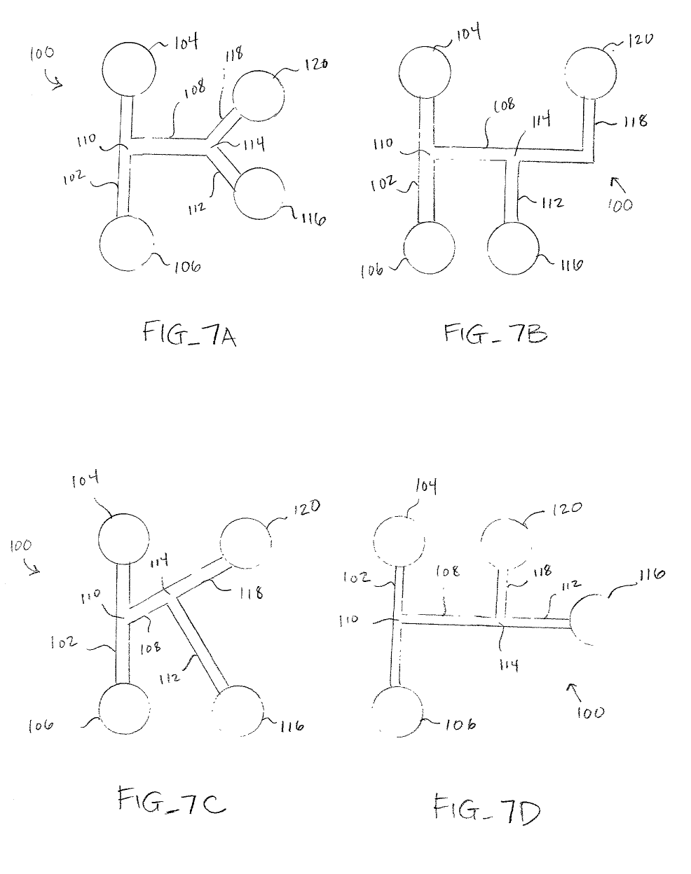

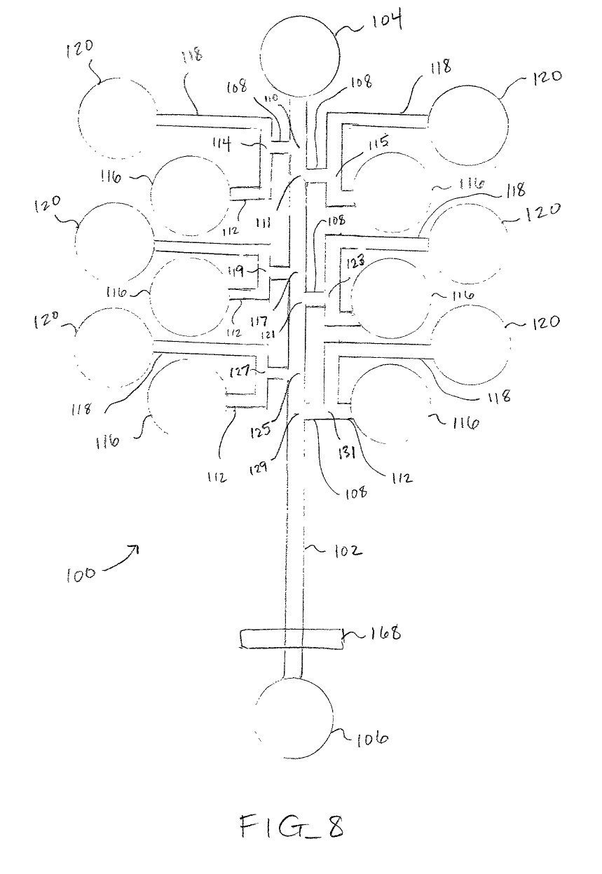

As previously mentioned, the channels may intersect in a variety of configurations while maintaining the essence of the invention. FIGS. 7A-7D illustrate a number of these configurations. For example, the loading channel 112 and the waste channel 118 may intersect the injection channel 108 at any angle to form the second intersection 114. FIG. 7A illustrates the channels 112,118 intersecting at approximately a 45 degree angle. Alternatively, as shown in FIG. 7B, the waste channel 118 may be configured so that the loading channel 112 and portions of the waste channel 118 are parallel. Here, the loading channel 112 and waste channel 118 still intersect the injection channel 108 at the second intersection 114. Referring to FIG. 7C, the system 100 may have a “K” configuration in which the injection channel 108 intersects the analysis channel 102 at an angle which is less than 90 degrees. Here the waste channel 118 is aligned with the injection channel 108 and the loading channel 112 intersect the injection channel 108 at a 90 degree angle at the second intersection 114. Alternatively, as shown in FIG. 7D, the loading channel 112 is aligned with the injection channel 108. The waste channel 118 intersects the injection channel 108 at the second intersection 114.

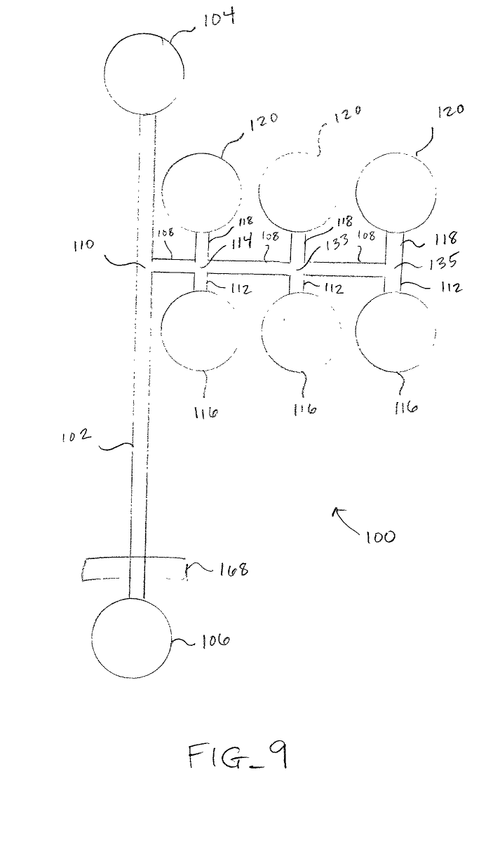

In addition, as shown in FIG. 8, the microfluidic system 100 of the present invention may comprise more than one injection channel 108 intersecting the analysis channel 102. As shown in the upper left of FIG. 8, one injection channel 108 intersects the analysis channel 102 at the first intersection 110. The loading channel 112 and waste channel 118 intersect the injection channel 108 at the second intersection 114. Opposite this set of channels, another injection channel 108 intersects the analysis channel 102 at a third intersection 111. The loading channel 112 and waste channel 118 intersect the injection channel 108 at a fourth intersection 115. This pattern continues with a fifth intersection 117, sixth intersection 119, seventh intersection 121, eighth intersection 123, ninth intersection 125, tenth intersection 127, eleventh intersection 129 and twelfth intersection 131. Thus, sample plugs can be simultaneously prepared, loaded and injected into intersections 110, 111, 117, 121, 125, 129 for analysis in the analysis channel 102. It may be appreciated that any number of injection channels 108 may intersect the analysis channel 102 and the channels 112, 118 and wells 116, 120 which are fluidly connected with the injection channels 108 may have any configuration as previously described.

Further, as shown in FIG. 9, the microfluidic system 100 of the present invention may comprise more than one set of loading channels 112/waste channels 118 intersecting the injection channel 108. As shown to the immediate right of the analysis channel 102, the loading channel 112 and waste channel 118 intersect the injection channel 108 at the second intersection 114. Further to the right, another loading channel 112 and waste channel 118 intersect the injection channel 108 at a third intersection 133. And, another loading channel 112 and waste channel 118 intersect the injection channel 108 at a fourth intersection 135. Thus, sample plugs can be simultaneously prepared and loaded into intersections 114, 133, 135. The sample plugs can then be injected into the analysis channel 102 together. It may be appreciated that any number of loading channel 112/waste channel 118 sets may intersect the injection channel 108 and the channels 112, 118 and wells 116, 120 may have any configuration as previously described. It may further be appreciated that the embodiments illustrated in FIG. 8 and FIG. 9 may be combined. Thus, it may be appreciated that a number of channel configurations are within the scope of the present invention.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

What is claimed is:

1. A microfluidic system comprising:
a structure;
an analysis channel within the structure;
an injection channel within the structure which intersects the analysis channel at a three-way first intersection;
a loading channel and a waste channel within the structure intersecting the injection channel at a second intersection; and
means for moving sample material through the injection channel to the analysis channel.

2. A system as in claim 1, further comprising means for moving sample material through the second intersection from the loading channel to the waste channel.

3. A system as in claim 2, further comprising a sample well fluidly connected to the loading channel and a waste well fluidly connected to the waste channel.

4. A system as in claim 3, wherein means for moving sample material through the second intersection from the loading channel to the waste channel comprises at least one electrode positioned within the sample well and/or the waste well which applies a voltage differential across at least one channel.

5. A system as in claim 4, wherein the waste well has a more positive electrode.

6. A system as in claim 4, wherein the waste well has a more negative electrode.

7. A system as in claim 3, wherein means for moving sample material through the second intersection from the loading channel to the waste channel comprises at least one pump or vacuum connected with the sample well and/or the waste well which applies a pressure differential across at least one channel.

8. A system as in claim 1, further comprising means for moving sample material through the injection channel from the second intersection to the first intersection.

9. A system as in claim 8, further comprising a sample well fluidly connected to the loading channel and a first well and a second well each fluidly connected to the analysis channel, and wherein the means for moving sample material through the injection channel from the second intersection to the first intersection comprises at least one electrode positioned within at least the sample well and the first well or the second well which applies a voltage differential across at least one channel.

10. A system as in claim 9, wherein the first well or second well has a more positive electrode

11. A system as in claim 9, wherein the first well or second well has a more negative electrode.

12. A system as in claim 8, further comprising a sample well fluidly connected to the loading channel and a first well and a second well each fluidly connected to the analysis channel, and wherein the means for moving sample material through the injection channel from the second intersection to the first intersection comprises at least one pump or vacuum connected with the sample well and/or the waste well which applies a pressure differential across at least one channel.

13. A system as in claim 1, wherein the analysis channel comprises an electrophoretic separation channel.

14. A system as in claim 13, further comprising a detector.

15. A system as in claim 14, wherein the electrophoretic separation channel and the detector reside between the first intersection and the second well.

16. A system as in claim 1, wherein the injection channel intersects the analysis channel at a 90 degree angle.

17. A system as in claim 1, wherein the injection channel intersects the analysis channel at a 45 degree angle.

18. A system as in claim 1, wherein at least the loading channel or the waste channel are parallel to the analysis channel.

19. A system as in claim 1, wherein the loading channel or the waste channel are aligned with the injection channel.

20. A system as in claim 1, further comprising:
another injection channel within the structure which intersects the analysis channel at a three-way third intersection; and
another loading channel and another waste channel within the structure intersecting the injection channel at a fourth intersection; and
means for moving sample material through the another injection channel to the analysis channel.

21. A system as in claim 20, further comprising another sample well fluidly connected to the another loading channel and another waste well fluidly connected to the another waste channel.

22. A system as in claim 21, wherein means for moving sample material through the fourth intersection from the another loading channel to the another waste channel comprises at least one electrode positioned within the another sample well and/or the another waste well which applies a voltage differential across at least one channel.

23. A system as in claim 1, further comprising:
another loading channel and another waste channel within the structure intersecting the injection channel at a third intersection; and
means for moving sample material through the third intersection to the analysis channel.

24. A system as in claim 23, further comprising another sample well fluidly connected to the another loading channel and another waste well fluidly connected to the another waste channel.

25. A system as in claim 24, wherein means for moving sample material through the third intersection to the analysis channel comprises at least one electrode positioned within the another sample well and/or the another waste well which applies a voltage differential across at least one channel.

26. A method for moving sample material within a microfluidic system, said method comprising:
providing the microfluidic system wherein the system comprises a structure having an analysis channel, an injection channel which intersects the analysis channel at a three-way first intersection, and a loading channel and a waste channel intersecting the injection channel at a second intersection; and
applying an injection force to move the sample material along the injection channel and into the analysis channel.

27. A method as in claim 26, further comprising applying a loading force to move sample material along the loading channel to the waste channel.

28. A method as in claim 27, wherein the microfluidic system further comprises a sample well fluidly connected to the loading channel and a waste well fluidly connected to the waste channel, and wherein applying the loading force comprises applying a voltage differential between the sample well and waste well.

29. A method as in claim 28, wherein the voltage differential comprises 200-400 volts.

30. A method as in claim 27, wherein the microfluidic system further comprises a sample well fluidly connected to the loading channel and a waste well fluidly connected to the waste channel, and wherein applying the loading force comprises applying a pressure differential between the sample well and waste well.

31. A method as in claim 27, further comprising removing the loading force when a desired portion of the sample material is located within the second intersection.

32. A method as in claim 26, wherein the microfluidic system further comprises a sample well fluidly connected to the loading channel and a first well and a second well fluidly connected to the analysis channel, and wherein applying the injection force comprises applying a voltage differential between the first well or second well and the sample well.

33. A method as in claim 26, wherein the microfluidic system further comprises a sample well fluidly connected to the loading channel and a first well and a second well fluidly connected to the analysis channel, and wherein applying the injection force comprises applying a pressure differential between the first well or second well and the sample well.

34. A method as in claim 26, further comprises removing the injection force when a desired portion of the sample material has entered or passed through the first intersection.

35. A method as in claim 35, wherein removing the injection force occurs when the desired portion of the sample material has moved along the analysis channel.

36. A method as in claim 35, wherein removing the injection force occurs 1-10 seconds after applying the injection force.

37. A method as in claim 26, further comprising applying a withdrawal force to move the sample material along the injection channel and into the waste channel.

38. A method as in claim 26, further comprising applying a voltage differential across the analysis channel to perform electrophoretic separation of sample material within the analysis channel.

39. A method for moving sample material within a microfluidic system, said method comprising:
providing the microfluidic system wherein the system comprises a structure having an analysis channel, an injection channel which intersects the analysis channel at a first intersection, and a loading channel and a waste channel intersecting the injection channel at a second intersection; and
applying a loading force to move the sample material along the loading channel to the second intersection;
simultaneously applying an analysis force to analyze sample material within the analysis channel.

40. A method as in claim 39, further comprising applying an injection force after applying the loading force to move the sample material from the second intersection into the analysis channel.

41. A method as in claim 39, wherein the system further comprises another injection channel within the structure which intersects the analysis channel at a third intersection, and another loading channel and another waste channel within the structure intersecting the injection channel at a fourth intersection, the method further comprising simultaneously applying another loading force to move sample material along the another loading channel to the fourth intersection.

42. A method as in claim 41, further comprising applying at least one injection force after applying the loading forces to move sample material from the second intersection and the fourth intersection into the analysis channel.

43. A method as in claim 39, wherein the system further comprises another loading channel and another waste channel within the structure intersecting the injection channel at a third intersection, the method further comprising simultaneously applying another loading force to move sample material along the another loading channel to the third intersection.

44. A method as in claim 43, further comprising applying at least one injection force after applying the loading forces to move sample material from the second intersection and the third intersection into the analysis channel.
